(19) United States
(12) Patent Application Publication
(10) Pub. No.: US 2010/0055171 A1
(43) Pub. Date: Mar. 4, 2010

(54) Pharmaceutical Formulation Comprising Donepezil

(75) Inventors: Violeta Cindric, Zagreb (HR); Ivica Grebenar, Zagreb (HR); Snjezana Miric, Zagreb (HR); Maja Devcic, Zagreb (HR)

Correspondence Address: STERNE, KESSLER, GOLDSTEIN & FOX P.L.L.C., 1100 NEW YORK AVENUE, N.W., WASHINGTON, DC, 20005, US

(73) Assignee: PLIVA Hrvatska d.o.o., Zagreb (HR)

(21) Appl. No.: 12/374,786

(22) PCT Filed: Jun. 29, 2007
(86) PCT No.: PCT/GB2007/002452

§ 371 Date: Nov. 6, 2009

(30) Foreign Application Priority Data
Jul. 22, 2006 (GB) 0614586.6

(51) Int. Cl. A61K 9/48; A61K 31/445; A61K 9/28

(52) U.S. Cl. 424/451; 514/319; 424/474

(57) ABSTRACT

The present invention relates to a pharmaceutical composition comprising 2,3-dihydro-5,6-dimethoxy-2-[[1-(phenylmethyl)-4-piperidinyl]methyl]-1H-inden-1-one, herein after referred to as Donepezil, or a pharmaceutically acceptable salt thereof, for treating of Alzheimer's disease and senile dementia. The present invention also relates to a manufacturing process for the preparation of Donepezil tablets containing and retaining polymorphic Form I of Donepezil hydrochloride. This composition also discloses the use of the excipients that ensure adequate flowability of a dry blend as well as required content uniformity and drug release rate of the final product.

The present invention relates to a pharmaceutical composition comprising 2,3-dihydro-5,6-dimethoxy-2-[[1-(phenylmethyl)-4-piperidinyl]methyl]-1H-inden-1-one, herein after referred to as Donepezil, or a pharmaceutically acceptable salt thereof, for treating of Alzheimer's disease and senile dementia. The present invention also relates to a manufacturing process for the preparation of Donepezil tablets containing and retaining polymorphic Form I of Donepezil hydrochloride. This composition also discloses the use of the excipients that ensure adequate flowability of a dry blend as well as required content uniformity and drug release rate of the final product.

### BACKGROUND OF THE INVENTION

Alzheimer's disease (AD) is an irreversible, progressive disorder in which brain cells (neurons) deteriorate, resulting in the loss of cognitive functions, primarily memory, judgment and reasoning, movement coordination, and pattern recognition. In advanced stages of the disease, all memory and mental functioning may be lost. A person with Alzheimer's disease has problems with memory, judgment, and thinking, which makes it hard for the person to work or take part in day-to-day life. The death of the nerve cells occurs gradually over a period of years. It is associated with senile dementia which is the mental deterioration (loss of intellectual ability) that is associated with old age. Two major types of senile dementia are identified: those due to generalized atrophy (Alzheimer type) and those due to vascular problems (mainly strokes). Senile dementia is often used when referring to Alzheimer's disease. Alzheimer's disease is most likely to affect older people: of all people over 80, 20% suffers from Alzheimer's disease.

There is currently no cure for Alzheimer's disease, although there are drugs which offer symptomatic benefit, specifically with respect to short-term memory impairment. Acetylcholinesterase inhibition was thought to be important because there is selective loss of forebrain cholinergic neurons as a result of Alzheimer's. AChE-inhibitors reduce the rate at which acetylcholine (ACh) is broken down and hence increase the concentration of ACh in the brain (combating the loss of ACh caused by the death of the cholinergic neurons). Acetylcholinesterase-inhibitors seemed to modestly moderate symptoms but do not prevent disease progression including cell death.

Donepezil hydrochloride is known to have such acetylcholinesterase inhibition properties. Donepezil and its salts, have application in treatment of a variety of disorders, including senile dementia and attention deficit disorder. In particular Donepezil hydrochloride is employed as a pharmaceutically active agent for the symptomatic treatment of mild to moderate Alzheimer's dementia and is currently formulated as film coated tablets of 5 milligram (mg) and 10 milligram (mg) doses for once a day oral administration under trade name Aricept.

WO 97/46527 describes a method for the preparation of the polymorphic forms I to V and of the amorphous form of Donepezil hydrochloride. Different methods for producing the Form I of Donepezil hydrochloride are described but no specific solid pharmaceutical formulations are disclosed. Also stability of amorphous or polymorphic forms of Donepezil hydrochloride was not elaborated.

EP 378238 A1 describes pharmaceutical compositions which comprise Donepezil hydrochloride in amorphous form. It is further discussed in this document that it is not an easy task to reproducibly prepare formulations including the desired polymorphic form of Donepezil hydrochloride since it is showing polymorphism and since similar procedures may nevertheless lead to different crystalline forms.

WO 2006/045512 describes pharmaceutical formulation which comprises Donepezil hydrochloride in polymorphic Form I or IV and has a content of water of 3 to 10% by weight (determined by Karl-Fischer). This document also discusses the content of water in the final formulation and its importance for stability of polymorphic forms present in the formulation. The document further states that it is crucial to control the content of water to lie solely within the above stated range in order to prevent the undesired conversion of the specific polymorphic form of Donepezil hydrochloride in the formulation to other hydrated or anhydrous polymorphic forms.

### DESCRIPTION OF THE INVENTION

Only one example of the prior art deals with the problem of avoiding a conversion of polymorphic forms of Donepezil hydrochloride during processing into the desired solid composition or during prolonged storage. According to the cited document it is only possible to prevent an undesired conversion of the specific polymorphic form of Donepezil hydrochloride by controlling a content of water in between of 3 and 10% by weight (determined by Karl-Fischer).

Within this invention it has been surprisingly found that prevention of the undesired conversion of the specific polymorphic form of Donepezil hydrochloride and therefore the desired stability of the final pharmaceutical composition can also be achieved with a content of water of less than 3% by weight, preferably less than 2% by weight, more preferably less than 1.8% by weight, even more preferably less than 1.6% by weight (determined by Karl-Fischer).

According to the invention we provide pharmaceutical composition comprising donepezil or a pharmaceutically acceptable salt (preferably hydrochloride) thereof in the form of a hydrate, the pharmaceutical composition having a content of water of less than 3% by weight (determined by Karl-Fischer)

An object of the present invention is to provide a stable pharmaceutical composition of Donepezil hydrochloride preferably monohydrate in which specific polymorphic form of Donepezil hydrochloride is stable and does not convert to other polymorphic forms (e.g. Form II or Form III) during formulation. Donepezil hydrochloride within the composition is preferably of Form I. It is a further object of the present invention to provide the given pharmaceutical composition in one of following forms; tablet (optionally with applied film coating), a capsule, pellets or in form of mini tablets filled in capsule. The preferred form is that of a tablet and more preferably in form of a film coated tablet. Tablet can be of round or oval biconvex shape with optionally scored or debossed sides if desired. The preferred shape is round. It is a further object of the present invention to provide a tablet formulation of the given composition comprising Donepezil hydrochloride with a content of water of less than 3% by weight. It is a further object of the present invention to provide a tablet formulation of Donepezil hydrochloride in which the tablet may readily be manufactured and does not demonstrate manufacturing problems such as capping, lamination, segregation (inhomogeneity) and poor flow characteristics.

Desired formulation of the given composition can be obtained by commonly used technologies: dry granulation, wet granulation and direct compression. Preferably it is done by means of direct compression. According to the direct compression method a manufacturing process includes following steps:
1. homogenization of active substance and excipients to obtain tabletting blend
2. compression of tabletting blend into tablet cores and
3. optionally, film coating of tablet cores

A procedure of obtaining a tabletting blend is performed in a specific way that gives adequate quality of the final product in terms of homogeneity and uniformity of tabletting blend by mixing under the specified process parameters. The tabletting blend is afterwards processed on rotary tablet press under set conditions in order to obtain tablet cores of specified characteristics (e.g. appropriate hardness that ensures low friability of tablet cores and enables satisfactory film coating). Direct compression method has the advantage of employing the least amount of operational manipulation, the key running powder requirements (blend homogeneity, consistent bulk density, flow and compressibility) must be met by the dry blend of active agent with excipients as there is no chemical or physical modification before tabletting.

Produced tablet cores can optionally be subjected to a film coating process with conventional materials used for film coating (as described in Pharmaceutical Coating Technology, 1995.). Film coating is a process wherein tablet cores are tumbled in coating pan while heating and applying film coating material are performed. Therefore appropriate friability is essential for maintaining integrity of tablets and thereby acceptable appearance of final product.

According to the invention we provide a pharmaceutical composition comprising donepezil or a pharmaceutically acceptable salt (preferably hydrochloride) thereof in the form of a hydrate, the pharmaceutical composition having a content of water of less than 3% by weight (determined by Karl-Fischer).

The filler, which can be selected from one or more of the following; lactose monohydrate, lactose anhydrate, starch, sugar or sugar alcohols (such as glucose, sucrose, sorbitol, mannitol), celluloses (in powder forms of different types (e.g. microcrystalline cellulose)), and dicalcium phosphate dihydrate.

The lactose may be in the form of lactose monohydrate or lactose anhydrate, but will preferably be lactose anhydrate. The lactose may be crystalline or amorphous. Suitably the lactose may be spray-dried (e.g. spray dried lactose anhydrate, e.g. Pharmatose™ DCL 22). Starch may for example be corn starch (e.g. unmodified corn starch or alternatively pre-gelatinised corn starch). The starch may also convey some disintegrant properties to the formulation. The total amount of filler present in the final composition is 50 to 92% by weight, preferably 65 to 90% by weight.

The binder, which can be selected from one or more of the following; hydroxypropyl cellulose, hydroxypropylmethyl cellulose or other cellulose ethers, vinylpyrrolidone containing polymers. Preferably it will be hydroxypropyl cellulose. Suitably the cellulose will be present in the final formulation at a concentration of 1 to 10% by weight, preferably 2 to 5% by weight, more preferably 3 to 4% by weight. The vinylpyrrolidone containing polymer may for example be polyvinylpyrrolidone alone or a mixture of polyvinylpyrrolidone and a co-polymer of vinylpyrrolidone and vinyl acetate or a co-polymer of vinylpyrrolidone and vinyl acetate alone.

The binder and the filler combined are present in the final formulation at a concentration of 50 to 97%, preferably 70 to 95% by weight, more preferably 80 to 93% by weight.

The antiadherent which can be selected from one or more of the following; colloidal silicon dioxide (e.g. Aerosil™ 200) or talc. Preferably the antiadherent is colloidal silicon dioxide. Antiadherent is added to the composition in order to improve the flow and packing properties of the composition. Antiadherent will be included in an amount of 0.1 to 5% by weight, preferably 0.5 to 1.5% by weight.

The disintegrant which can be selected from one or more of the following; crospovidone (cross linked polyvinylpyrrollidone), sodium starch glycolate, croscarmellose sodium, powdered cellulose, microcrystalline cellulose or carboxymethylcellulose calcium. Preferably disintegrant is sodium starch glycolate.

Disintegrant is added to the composition to enhance the disintegrating properties of the composition and thereby to accelerate dissolution. Disintegrant will be included in an amount of 1 to 10% by weight, preferably 3 to 8% by weight, more preferably 5 to 7% by weight.

The lubricant which can be selected from one or more of the following; stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc. Preferably lubricant is magnesium stearate. Lubricant will be included in an amount of 0.1 to 5% by weight preferably 0.5 to 1.5% by weight.

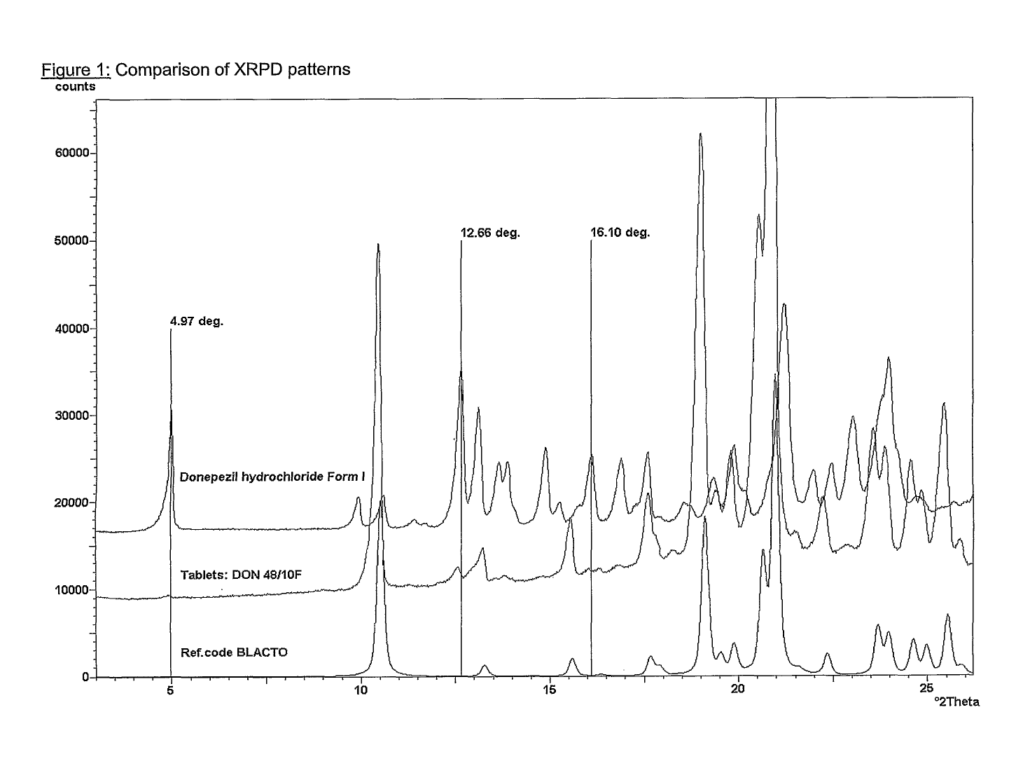

The desired stability of polymorphic form within the described composition, with the content of water in total amount of less than 3% by weight, is achieved as shown in FIG. 1. FIG. 1 shows the results of the XRPD analysis performed on samples of the final product of the described composition.

Samples subjected to analysis for which results are shown in FIG. 1:
1. Ref. code BLACTO—calculated XRPD pattern of anhydro lactose (β lactose). Data taken from Cambridge Structural Database version 5.27, K. Hirotsu, A. Shimada Bull. Chem. Soc. Jpn. 47 (1974) 1872.
2. Donepezil hydrochloride Form I
3. Final formulation (film tablets) of donepezil: DON 48/10F

### EXAMPLE 1

| COMPOSITION OF A TABLET | mg/tbl |
|---|---|
| Donepezil hydrochloride | 10.00 |
| Starch | 28.00 |
| Lactose anhydrous | 219.00 |
| Hydroxypropyl cellulose | 8.00 |
| Sodium starch glycolate | 11.00 |
| Colloidal silicon dioxide | 1.50 |
| Magnesium stearate | 2.00 |
| Lactose monohydrate | 3.00 |
| Hydroxypropylmethyl cellulose | 2.00 |
| Polyethylene glycole | 1.00 |
| Titanium dioxide | 2.00 |
| Iron oxide yellow | 0.50 |

Preparation of Tablet Core:

Donepezil hydrochloride was mixed with starch, lactose anhydrous, hydroxypropyl cellulose, sodium starch glycolate and colloidal silicon dioxide and homogenized for 25 minutes.

Magnesium stearate, screened through a 0.6 mm sieve, was added to the core component above. The final blend was homogenised for additional 5 minutes and then compressed into tablets.

Coating:

The tablets were coated with aqueous suspension of lactose monohydrate, hydroxypropylmethyl cellulose, polyethylene glycol, titanium dioxide and iron oxide yellow

### EXAMPLE 2

| COMPOSITION OF A TABLET | mg/tbl |
|---|---|
| Donepezil hydrochloride | 10.00 |
| Lactose anhydrous | 245.00 |
| Hydroxypropyl cellulose | 7.20 |
| Sodium starch glycolate | 8.20 |
| Colloidal silicon dioxide | 2.10 |
| Sodium stearyl fumarate | 2.50 |

Donepezil hydrochloride was homogenised with lactose anhydrous, hydroxypropyl cellulose, sodium starch glycolate and colloidal silicon dioxide for 20 minutes. Sodium stearyl fumarate, screened through a 0.6 mm sieve, was added to the core component above. The final blend was homogenised for additional 5 minutes and then compressed into tablets. The tablets were filled in the capsules.

### EXAMPLE 3

| COMPOSITION OF A TABLET | mg/tbl |
|---|---|
| Donepezil hydrochloride | 10.00 |
| Starch | 28.00 |
| Lactose anhydrous | 219.00 |
| Hydroxypropyl cellulose | 9.00 |
| Sodium starch glycolate | 10.00 |
| Colloidal silicon dioxide | 1.50 |
| Magnesium stearate | 2.00 |
| Lactose monohydrate | 3.00 |
| Hydroxypropylmethyl cellulose | 2.00 |
| Polyethylene glycol | 1.00 |
| Titanium dioxide | 2.00 |
| Iron oxide yellow | 0.50 |

Preparation of Tablet Core:

Donepezil hydrochloride and hydroxypropyl cellulose were homogenized for 10 minutes and compressed into tablets. The tablets were afterwards milled and sieved through suitable sieve and added to the other substances, except magnesium stearate.

Magnesium stearate, screened through a 0.6 mm sieve, was added to the core component above. The final blend was homogenised for additional 5 minutes and then compressed into tablets.

Coating:

The tablets were coated with aqueous suspension of lactose monohydrate, hydroxypropylmethyl cellulose, polyethylene glycole, titanium dioxide and iron oxide yellow

1-23. (canceled)

24. A pharmaceutical composition comprising donepezil or a pharmaceutically acceptable salt thereof in the form of a hydrate, the pharmaceutical composition having a content of water of less than 3% by weight (determined by Karl-Fischer).

25. The composition of claim 24 comprising from 2% to 10% by weight of donepezil.

26. The composition of claim 24, wherein donepezil is donepezil hydrochloride.

27. The composition of claim 26, wherein donepezil hydrochloride is of crystalline form.

28. The composition of claim 26, wherein donepezil hydrochloride is of polymorphic Form I.

29. The composition of claim 26, wherein donepezil hydrochloride is donepezil hydrochloride monohydrate.

30. The composition of claim 24, further comprising one or more pharmaceutically acceptable excipients selected from the group consisting of a binder, filler, disintegrant, lubricant, antiadherent, and combinations thereof.

31. The composition of claim 30, wherein said antiadherent is selected from the group consisting of colloidal silicon dioxide, talc, and combinations thereof.

32. The composition of claim 31, comprising from 0.1% to 5% by weight of colloidal silicon dioxide.

33. The composition of claim 30, wherein said filler is selected from the group consisting of lactose monohydrate, lactose anhydrate, starch, and combinations thereof.

34. The composition of claim 33, comprising from 50% to 92% by weight of filler.

35. The composition of claim 30, wherein said binder is selected from the group consisting of microcrystalline cellulose, hydroxypropyl cellulose, hydroxypropylmethyl cellulose, vinylpyrrolidine containing polymer, starch, and combinations thereof.

36. The composition of claim 35, comprising from 1% to 10% by weight of hydroxypropyl cellulose.

37. The composition of claim 30, wherein said disintegrant is selected from the group consisting of crospovidone (cross-linked polyvinylpyrrolidone), sodium starch glycolate, croscarmellose sodium, powdered cellulose, microcrystalline cellulose, carboxymethylcellulose calcium, and combinations thereof.

38. The composition of claim 37, comprising from 0.5% to 10% by weight of sodium starch glycolate.

39. The composition of claim 30, wherein said lubricant is selected from the group consisting of stearic acid, metal salt stearates, sodium stearyl fumarate, sodium lauryl sulphate, sodium benzoate, glyceryl behenate, glyceryl monostearate, glyceryl palmitostearate, polyethylene glycol, hydrogenated vegetable oil, talc, and combinations thereof.

40. The composition of claim 39, comprising from 0.1 to 5% by weight of a metal salt stearate, wherein the metal salt stearate is magnesium stearate.

41. The composition of claim 24, in the form of a tablet or in form of a capsule or in form of pellets or in form of mini tablets filled in a capsule.

42. The composition of claim 41, in the form of tablet.

43. The composition of claim 42, in the form of a film coated tablet.

44. The composition of claim 43, comprising from 2% to 5% by weight of a film coating.

45. A process for manufacturing the composition of claim 24, comprising:
a) homogenizing an active substance and one or more excipients to obtain a tabletting blend;
b) compressing the tabletting blend into tablet cores;
c) optionally film coating the tablet cores; and
d) optionally filling the coated or uncoated tablets in one or more capsules.

46. A pharmaceutical composition, comprising donepezil or a pharmaceutically acceptable salt thereof in the form of a hydrate, lactose anhydrous, hydroxypropyl cellulose, sodium starch glycolate, and colloidal silicon dioxide, wherein the pharmaceutical composition has a content of water of less than 3% by weight (determined by Karl-Fischer).

47. The pharmaceutical composition of claim 46, further comprising starch, lactose monohydrate, hydroxypropylmethyl cellulose, magnesium stearate, and polyethylene glycol.

48. The pharmaceutical composition of claim 46, further comprising sodium stearyl fumarate.
